(19) United States
(12) Patent Application Publication
(10) Pub. No.: US 2024/0074733 A1
(43) Pub. Date: Mar. 7, 2024

(54) APPARATUS, SYSTEM AND METHOD TO CONTROL AN ULTRASONIC IMAGE ON A DISPLAY BASED ON SENSOR INPUT AT AN ULTRASONIC IMAGING DEVICE

(71) Applicant: Exo Imaging, Inc., Redwood City, CA (US)

(72) Inventors: Tanya L. Frane, Phoenix, AZ (US); Sandeep Akkaraju, Wellesley, MA (US); Brian Bircumshaw, Orinda, CA (US); Janusz Bryzek, Oakland, CA (US); Arun Nagdev, San Francisco, CA (US)

(73) Assignee: Exo Imaging, Inc., Redwood City, CA (US)

(21) Appl. No.: 17/901,461

(22) Filed: Sep. 1, 2022

(51) Int. Cl. A61B 8/00

(52) U.S. Cl. CPC A61B 8/467; A61B 8/4254

(57) ABSTRACT

An apparatus, a method, and computer-implemented media. The apparatus is to receive information based on haptic input to a surface of a housing of an ultrasound imaging device; and based on the information, execute an ultrasound exam function corresponding to the haptic input, the ultrasound exam function to control an ultrasound image on a display of a computing system.

### FIELD

Embodiments relate in general to the field of ultrasonic imaging devices.

### BACKGROUND

Ultrasound imaging is widely used in the fields of medicine and non-destructive testing and may have a diagnostic or a procedural purpose. While a diagnostic ultrasound examination can involve imaging without performing a procedure on a patient being subjected to the examination, a procedural ultrasound involves a complex examination where, in addition to use of an ultrasonic prove for imaging, the user inserts a medical instrument, such as a needle or catheter, into tissue. Procedural ultrasound requires small fine movements of both the needle and the ultrasonic probe, and both procedural and diagnostic ultrasound require controlled movements of the ultrasonic probe in order to capture the needed images. The user typically captures images during an ultrasound examination, as well as making fine-tuned adjustments to the ultrasound image generated on a display of a computing system. A user typically uses one hand to hold and guide the ultrasonic probe, while using his/her other hand to operate a user interface associated with the ultrasonic probe in order to control ultrasound exam functions such as freezing or saving ultrasound images on a display. Where a user does not have personnel to assist during an ultrasound exam, the state of the art provides either a physical push button at a fixed region of the ultrasound probe housing, or a foot pedal, or voice, or VR headset, and this in order to allow ultrasound exam functions to be controlled by the user during the ultrasound exam process. Physical push buttons of the state of the art require the user to change his/her grip during an ultrasound exam in order to be able to control ultrasound exam functions, which can move the probe and negatively affect the ultrasound image being generated therefrom by altering a set ultrasound image location that the computing system expects. The foot pedal solution on the other hand provides a bulky piece of hardware that needs to be attached to the ultrasound console of the computing system associated with the ultrasound device and is activated by stepping thereon to relay feedback to the computing system. The foot pedal option is thus cumbersome, and difficult to implement. Emerging Virtual Reality (VR) headsets aim at using eye movement.

### SUMMARY

The ultrasonic imaging device of some embodiments may operate according to one or more sets of instructions, using algorithms, either collectively or individually, to cause, by way of inertial movement of the ultrasound image device housing, execution of ultrasound exam functions relating to ultrasonic images on a display.

### BRIEF DESCRIPTION OF TW DRAWINGS

The novel features of embodiments are set forth with particularity in the appended claims. A better understanding of the features and advantages of Some embodiments will be obtained by reference to the following detailed description that sets forth illustrative embodiments, in which the principles of the invention are utilized, and the accompanying drawings (also “Figure” and “Fig.” herein), of which:

FIG. 1 is a block diagram of an ultrasound imaging device in accordance with some embodiments.

FIG. 2 is a diagram of an ultrasound imaging system in accordance with some embodiments.

FIG. 3 is a schematic diagram of an ultrasound imaging device in accordance with some embodiments.

FIGS. 4A and 4B, which show perspective views of a state of the art handheld ultrasonic probe being held in two different manners.

FIGS. 5A and 5B which show perspective views of a handheld ultrasonic probe according to an embodiment being held in two different manners

FIG. 6 shows a schematic illustration of an embodiment of sensor circuitry and of sensor signal processing circuitry according to an embodiment where both components are within a single package.

FIG. 7 depicts a flowchart of a process according to an embodiment.

FIG. 8 depicts a flowchart of a process according to another embodiment.

### DETAILED DESCRIPTION

Some embodiments advantageously provide an ultrasound imaging device, such as an ultrasonic probe, that includes sensor circuitry coupled to a housing thereof to sense inertial changes at the housing, and to cause, based on the sensed inertial changes, one or more ultrasound exam functions to be executed at a computing system associated with the ultrasound imaging device, the ultrasound exam functions including functions to control an ultrasound image on a display of the computing system.

The sensor circuitry may send information based on the sensed inertial changes to a processing circuitry, the processing circuitry to determine a correlation between the sensed inertial changes and one or more ultrasound exam functions to be executed at the computing system.

Ultrasound imaging devices, such as handheld ultrasound imaging devices, may require the use of three hands when scanning, using an associated operating interface, and using a medical device such as a needle or catheter in order to perform a procedure on a patient. Typically, one hand is used to guide the ultrasound imaging device during scanning, another hand is used to interact with the operating user interface, such as a computing system that includes an ultrasound display, and a third hand may be required to control a medical tool such as a needle or catheter on a patient during a procedural ultrasound.

Some embodiments advantageously allow a user to operate the user interface associated with an ultrasound imaging device without a need to change his/her grip on the ultrasound imaging device or without a need to move a finger along a height direction of the ultrasound imaging device, during a diagnostic or a procedural ultrasound examination.

Ultrasound imaging devices may be used to image internal tissue, bones, blood flow, or organs of human or animal bodies in a non-invasive manner. The images can then be displayed. To perform ultrasound imaging, the ultrasound imaging devices transmit an ultrasonic signal into the body and receive a reflected signal from the body part being imaged. Such ultrasound imaging devices include transducers and associated electronics, which may be referred to as transceivers or imagers, and which may be based on photo-acoustic or ultrasonic effects. Such transducers may be used for imaging and may be used in other applications as well. For example, the transducers may be used in medical imaging; flow measurements in arteries and pipes, can form speakers and microphone arrays; can perform lithotripsy; localized tissue heating for therapeutic; and highly intensive focused ultrasound (HIFU) surgery.

Additional aspects and advantages of some embodiments will become readily apparent to those skilled in this art from the instant detailed description, wherein only illustrative embodiments are shown and described. As will be realized, some embodiments are capable of achieving other, different goals, and their several details are capable of modifications in various obvious respects, all without departing from the disclosure. Accordingly, the drawings and description are to be regarded as illustrative in nature, and not as restrictive.

Traditionally, imaging devices such as ultrasound imagers used in medical imaging use piezoelectric (PZT) materials or other piezo ceramic and polymer composites. Such imaging devices may include a housing to house the transducers with the PZT material, as well as other electronics that form and display the image on a display unit. To fabricate the bulk PZT elements or the transducers, a thick piezoelectric material slab may be cut into large rectangular shaped PZT elements. These rectangular-shaped PZT elements may be expensive to build, since the manufacturing process involves precisely cutting generally the rectangular-shaped thick PZT or ceramic material and mounting it on substrates with precise spacing. Further, the impedance of the transducers is much higher than the impedance of the body tissue, which can affect performance.

Still further, such thick bulk PZT elements can require very high voltage pulses, for example 100 volts (V) or more to generate transmission signals. This high drive voltage can sometimes results in high power dissipation, since the power dissipation in the transducers is proportional to the square of the drive voltage. This high power dissipation generates heat within the ultrasound imaging device such that cooling arrangements are necessitated. These cooling systems increase the manufacturing costs and weights of the ultrasound imaging devices which makes the ultrasound imaging devices more burdensome to operate.

Some embodiments may be utilized in the context of imaging devices that utilize either piezoelectric micromachined ultrasound transducer (pMUT) or capacitive micromachine ultrasonic transducer (cMUT) technologies, as described in further detail herein.

In general, MUTs, such as both cMUT and pMUT, include a diaphragm (a thin membrane attached at its edges, or at some point in the interior of the probe), whereas a “traditional,” bulk PZT element typically consists of a solid piece of material.

Piezoelectric micromachined ultrasound transducers (pMUTs) may be efficiently formed on a substrate leveraging various semiconductor wafer manufacturing operations. Semiconductor wafers may currently come in 6 inch, 8 inch, and 12 inch sizes and are capable of housing hundreds of transducer arrays. These semiconductor wafers start as a silicon substrate on which various processing operations are performed. An example of such an operation is the formation of SiO2 layers, also known as insulating oxides. Various other operations such as the addition of metal layers to serve as interconnects and bond pads are performed to allow connection to other electronics. Yet another example of a machine operation is the etching of cavities. Compared to the conventional transducers having bulky piezoelectric material, pMUT elements built on semiconductor substrates are less bulky, are cheaper to manufacture, and have simpler and higher performance interconnection between electronics and transducers. As such, they provide greater flexibility in the operational frequency of the ultrasound imaging device using the same, and potential to generate higher quality images. Frequency response may for example be expanded though flexibility of shaping the diaphragm and its active areas with piezo material.

In some embodiments, the ultrasound imaging device includes an application specific integrated circuit (ASIC) that includes transmit drivers, sensing circuitry for received echo signals, and control circuitry to control various operations. The ASIC may be formed on the same or another semiconductor wafer. This ASIC may be placed in close proximity to pMUT or cMUT elements to reduce parasitic losses. As a specific example, the ASIC may be 50 micrometers (μm) or less away from the transducer array. In a broader example, there may be less than 100 μm separation between the 2 wafers or 2 die, where each wafer includes many die, and a die includes a transducer array in the transducer wafer and an ASIC array in the ASIC wafer. The array may have up to 10,000 or more individual elements. In some embodiments, the ASIC has matching dimensions relative to the pMUT or cMUT array and allows the devices to be stacked for wafer-to-wafer interconnection or transducer die on ASIC wafer or transducer die to ASIC die interconnection. Alternatively, the transducer can also be developed on top of the ASIC wafer using low temperature piezo material sputtering and other low temperature processing compatible with ASIC processing.

Wherever the ASIC and the transducer interconnect, according to one embodiment, the two may have similar footprints. More specifically, according to the latter embodiment, a footprint of the ASIC may be an integer multiple or divisor of the MUT footprint.

Regardless of whether the ultrasound imaging device is based on pMUT or cMUT, an imaging device according to some embodiments may include a number of transmit channels and a number of receive channels. Transmit channels are to drive the transducer elements with a voltage pulse at a frequencies the elements are responsive to. This causes an ultrasonic waveform to be emitted from the elements, which waveform is to be directed towards an object to be imaged (target object), such as toward an organ or other tissue in a body. In some examples, the ultrasound imaging device with the array of transducer elements may make mechanical contact with the body using a gel in between the ultrasound imaging device and the body. The ultrasonic waveform travels towards the object, i.e., an organ, and a portion of the waveform is reflected back to the transducer elements in the form of received/reflected ultrasonic energy where the received ultrasonic energy may converted to an electrical energy within the ultrasound imaging device. The received ultrasonic energy may be processed by a number of receive channels to convert the received ultrasonic energy to signals, and the signals may be processed by other circuitry to develop an image of the object for display based on the signals.

An embodiment of an ultrasound imaging device includes a transducer array, and control circuitry including, for example, an application-specific integrated circuit (ASIC), and transmit and receive beamforming circuitry, and optionally additional control electronics.

In an embodiment, an imaging device may include a handheld casing or handheld housing where transducers and associated electronic circuitries, such as a control circuitry and optionally a computing device are housed. The ultrasound imaging device may also contain a battery to power the electronic circuitries.

Thus, some embodiments pertain to a portable imaging device utilizing either pMUT elements or cMUT elements in a 2D array. In some embodiments, such an array of transducer elements is coupled to an application specific integrated circuit (ASIC) of the ultrasound imaging device.

In the following description, for purposes of explanation, specific details are set forth in order to provide an understanding of the disclosure. It will be apparent, however, to one skilled in the art that the disclosure may be practiced without these details. Furthermore, one skilled in the art will recognize that examples of the present disclosure, described below, may be implemented in a variety of ways, such as a process, one or more processors (processing circuitry) of a control circuitry, one or more processors (or processing circuitry) of a computing device, a system, a device, or a method on a tangible computer-readable medium.

One skilled in the art shall recognize: (1) that certain fabrication operations may optionally be performed; (2) that operations may not be limited to the specific order set forth herein; and (3) that certain operations may be performed in different orders, including being done contemporaneously, and (4) operations may involve using Artificial Intelligence.

Elements/components shown in diagrams are illustrative of exemplary embodiments and are meant to avoid obscuring the disclosure. Reference in the specification to “one example,” “preferred example,” “an example,” “examples,” “an embodiment,” “some embodiments,” or “embodiments” means that a particular feature, structure, characteristic, or function described in connection with the example is included in at least one example of the disclosure and may be in more than one example. The appearances of the phrases “in one example,” “in an example,” “in examples,” “in an embodiment,” “in some embodiments,” or “in embodiments” in various places in the specification are not necessarily all referring to the same example or examples. The terms “include,” “including,” “comprise,” and “comprising” shall be understood to be open terms and any lists that follow are examples and not meant to be limited to the listed items. Any headings used herein are for organizational purposes only and shall not be used to limit the scope of the description or the claims. Furthermore, the use of certain terms in various places in the specification is for illustration and should not be construed as limiting.

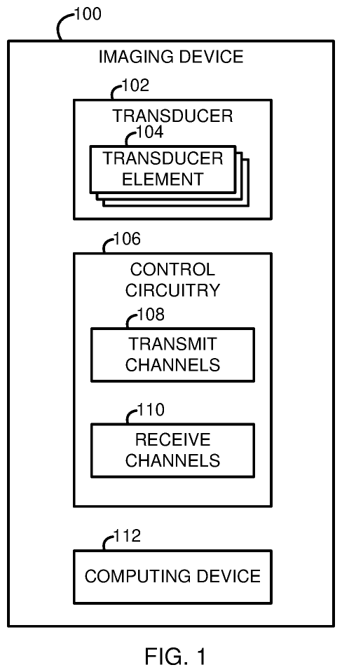

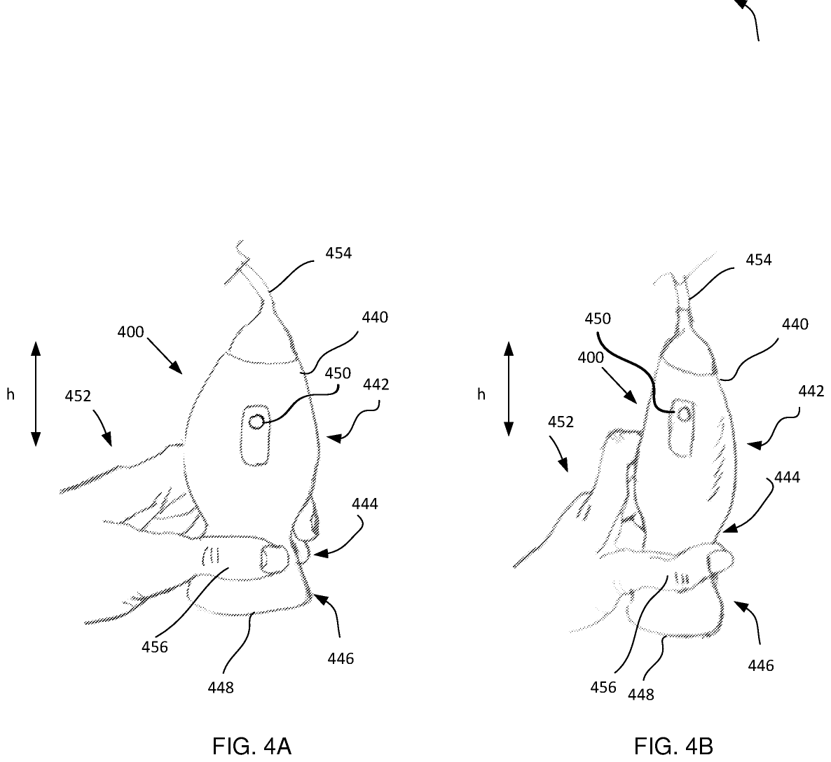

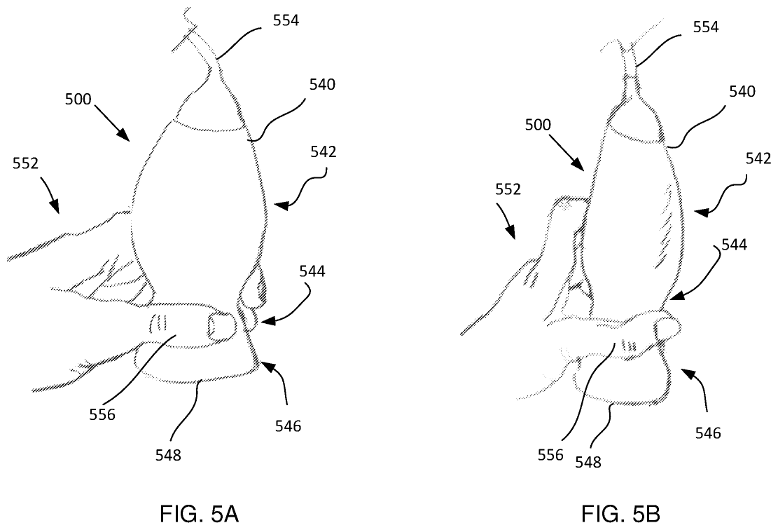

Reference will be made to FIGS. 1-3, which show devices and circuitries that may be used to implement some embodiments as described herein. Reference will further be made to FIGS. 4A and 4B which show an ultrasound imaging device according to the state of the art. Reference will thereafter be made to FIGS. 5A and 5B, which show an ultrasound imaging device according to one embodiment being held in two different manners.

Turning now to the figures, FIG. 1 is a block diagram of an imaging device 100 with a controller or control circuitry 106 controlling selectively alterable channels (108, 110) and having imaging computations performed on a computing device 112 according to principles described herein. As described above, the ultrasound imaging device 100 may be used to generate an image of internal tissue, bones, blood flow, or organs of human or animal bodies. Accordingly, the ultrasound imaging device 100 may transmit a signal into the body and receive a reflected signal from the body part being imaged. Such imaging devices may include either pMUT or cMUT, which may be referred to as transducers or imagers, which may be based on photo-acoustic or ultrasonic effects. The ultrasound imaging device 100 may be used to image other objects as well. For example, the ultrasound imaging device may be used in medical imaging; flow measurements in pipes, speaker, and microphone arrays; lithotripsy; localized tissue heating for therapeutic; and highly intensive focused ultrasound (HIFU) surgery.

In addition to use with human patients, the ultrasound imaging device 100 may be used to acquire an image of internal organs of an animal as well. Moreover, in addition to imaging internal organs, the ultrasound imaging device 100 may also be used to determine direction and velocity of blood flow in arteries and veins as in Doppler mode imaging and may also be used to measure tissue stiffness.

The ultrasound imaging device 100 may be used to perform different types of imaging. For example, the ultrasound imaging device 100 may be used to perform one-dimensional imaging, also known as A-Scan, two-dimensional imaging, also known as B scan, three-dimensional imaging, also known as C scan, and Doppler imaging (that is, the use of Doppler ultrasound to determine movement, such as fluid flow within a vessel). The ultrasound imaging device 100 may be switched to different imaging modes, including without limitation linear mode and sector mode, and electronically configured under program control.

To facilitate such imaging, the ultrasound imaging device 100 includes one or more ultrasound transducers 102, each transducer 102 including an array of ultrasound transducer elements 104. Each ultrasound transducer element 104 may be embodied as any suitable transducer element, such as a pMUT or cMUT element. The transducer elements 104 operate to 1) generate the ultrasonic pressure waves that are to pass through the body or other mass and 2) receive reflected waves (received ultrasonic energy) off the object within the body, or other mass, to be imaged. In some examples, the ultrasound imaging device 100 may be configured to simultaneously transmit and receive ultrasonic waveforms or ultrasonic pressure waves (pressure waves in short). For example, control circuitry 106 may be configured to control certain transducer elements 104 to send pressure waves toward the target object being imaged while other transducer elements 104, at the same time, receive the pressure waves/ultrasonic energy reflected from the target object, and generate electrical charges based on the same in response to the received waves/received ultrasonic energy/received energy.

In some examples, each transducer element 104 may be configured to transmit or receive signals at a certain frequency and bandwidth associated with a center frequency, as well as, optionally, at additional center frequencies and bandwidths. Such multi-frequency transducer elements 104 may be referred to as multi-modal elements 104 and can expand the bandwidth of the ultrasound imaging device 100. The transducer element 104 may be able to emit or receive signals at any suitable center frequency, such as about 0.1 to about 100 megahertz. The transducer element 104 may be configured to emit or receive signals at one or more center frequencies in the range from about 0.0.1 to about 100 megahertz.

To generate the pressure waves, the ultrasound imaging device 100 may include a number of transmit (Tx) channels 108 and a number of receive (Rx) channels 110. The transmit channels 108 may include a number of components that drive the transducer 102, i.e., the array of transducer elements 104, with a voltage pulse at a frequency that they are responsive to. This causes an ultrasonic waveform to be emitted from the transducer elements 104 towards an object to be imaged.

According to some embodiments, an ultrasonic waveform may include one or more ultrasonic pressure waves transmitted from one or more corresponding transducer elements of the ultrasound imaging device substantially simultaneously.

The ultrasonic waveform travels towards the object to be imaged and a portion of the waveform is reflected back to the transducer 102, which converts it to an electrical energy through a piezoelectric effect. The receive channels 110 collect electrical energy thus obtained, and process it, and send it for example to the computing device 112, which develops or generates an image that may be displayed.

In some examples, while the number of transmit channels 108 and receive channels 110 in the ultrasound imaging device 100 may remain constant, and the number of transducer elements 104 that they are coupled to may vary. A coupling of the transmit and receive channels to the transducer elements may be, in one embodiment, controlled by control circuitry 106. In some examples, for example as shown in FIG. 1, the control circuitry may include the transmit channels 108 and the receive channels 110. For example, the transducer elements 104 of a transducer 102 may be formed into a two-dimensional spatial array with N columns and M rows. In a specific example, the two-dimensional array of transducer elements 104 may have 128 columns and 32 rows. In this example, the ultrasound imaging device 100 may have up to 128 transmit channels 108 and up to 128 receive channels 110. In this example, each transmit channel 108 and receive channel 110 may be coupled to multiple or single pixels 104. For example, depending on the imaging mode (for example, whether a linear mode where a number of transducers transmit ultrasound waves in a same spatial direction, or a sector mode, where a number of transducers transmit ultrasound waves in different spatial directions), each column of transducer elements 104 may be coupled to a single transmit channel 108 and a single receive channel (110). In this example, the transmit channel 108 and receive channel 110 may receive composite signals, which composite signals combine signals received at each transducer element 104 within the respective column. In another example, i.e., during a different imaging mode, each transducer element 104 may be coupled to its dedicated transmit channel 108 and its dedicated receive channel 110. In some embodiments, a transducer element 104 may be coupled to both a transmit channel 108 and a receive channel 110. For example, a transducer element 104 may be adapted to create and transmit an ultrasound pulse and then detect the echo of that pulse in the form of converting the reflected ultrasonic energy into electrical energy.

The control circuitry 106 may be embodied as any circuit or circuits configured to perform the functions described herein. For example, the control circuitry 106 may be embodied as or otherwise include an application specific integrated circuit (ASIC), a field programmable gate array (FPGA), a system-on-a-chip, a processor and memory, a voltage source, a current source, one or more amplifiers, one or more digital-to-analog converters, one or more analog-to-digital converters, etc.

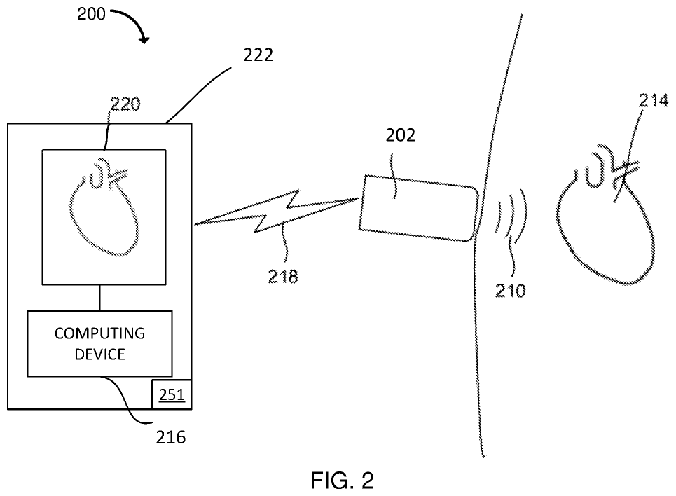

The illustrative computing device 112 may be embodied as any suitable computing device including any suitable components, such as one or more processors (i.e. one or more processing circuitries), one or more memory circuitries, one or more communication circuitries, one or more batteries, one or more displays, etc. In one embodiment, the computing device 112 may be integrated with the control circuitry 106, transducers 102, etc., into a single microelectronic package or single chip, or a single system on a chip (SoC), or a single ultrasound imaging device housing as suggested for example in the embodiment of FIG. 1. In other embodiments, some or all of the computing devices may be in a separate microelectronic package from the control circuitry, or in a separate device distinct from the ultrasound imaging device such as an ultrasound imaging probe, as suggested for example in the embodiment of in FIG. 2 as will be described in further detail below.

Each transducer element may have any suitable shape such as, square, rectangle, ellipse, or circle. The transducer elements may be arranged in a two dimensional array arranged in orthogonal directions, such as in N columns and M rows as noted herein or may be arranged in an asymmetric (or staggered) rectilinear array.

Transducer elements 104 may have associated transmit driver circuits of associated transmit channels, and low noise amplifiers of associated receive channels. Thus, a transmit channel may include transmit drivers, and a receive channel may include one or more low noise amplifiers. For example, although not explicitly shown, the transmit and receive channels may each include multiplexing and address control circuitry to enable specific transducer elements and sets of transducer elements to be activated, deactivated or put in low power mode. It is understood that transducers may be arranged in patterns other than orthogonal rows and columns, such as in a circular fashion, or in other patterns based on the ranges of ultrasonic waveforms to be generated therefrom.

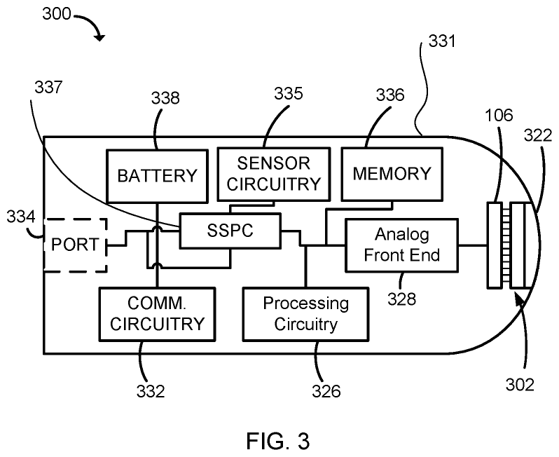

FIG. 2 is a diagram of an imaging environment including an imaging system 200 with selectively configurable characteristics, according to an embodiment. The imaging system of FIG. 2 may include an ultrasound imaging device 202 (which may be similar to ultrasound imaging device 300 described below in the context of FIG. 3) and a computing system 222 which includes a computing device 216 and a display 220 coupled to the computing device, as will be described in further detail below.

As depicted in FIG. 2, the computing device 216 may, according to one embodiment, and unlike the embodiment of FIG. 1, be physically separate from the ultrasound imaging device 220. For example, the computing device 216 and display device 220 may be disposed within a separate device (in this context, the shown computing system 222, physically separate from imaging device 202 during operation) as compared with the components of the ultrasound imaging device 202. The computing system 222 may include a mobile device, such as cell phone or tablet, or a stationary computing device, which can display images to a user. In another example, as shown in FIG. 1 for example, the display device, the computing device, and associated display, may be part of the ultrasound imaging device 202 (now shown). That is, the ultrasound imaging device 100, computing device 216, and display device 220 may be disposed within a single housing.

A “computing device” as referred to herein may, in some embodiments, be configured to generate signals to at least one of cause an image of the object to be displayed on a display, or cause information regarding the image to be communicated to a user.

A “computing device,” as referred to herein may, in some embodiments, be configured to receive sensor signals from sensor circuitry of an ultrasound imaging device, and to process those sensor signals to cause generation of execution signals to cause execution of ultrasound exam functions based on the sensor signals.

As depicted, the imaging system includes the ultrasound imaging device 202 that is configured to generate and transmit, via the transmit channels (FIG. 1, 108), pressure waves 210 toward an object, such as a heart 214, in a transmit mode/process. The internal organ, or other object to be imaged, may reflect a portion of the pressure waves 210 toward the ultrasound imaging device 202 which may receive, via a transducer (such as transducer 102 of FIG. 1), receive channels (FIG. 1, 110), control circuitry (FIG. 1, 106), the reflected pressure waves. The transducer may generate an electrical signal based on the received ultrasonic energy in a receive mode/process. A transmit mode or receive mode may be applicable in the context of imaging devices that may be configured to either transmit or receive, but at different times. However, as noted previously, some imaging devices according to embodiments may be adapted to be in both a transmit mode and a receive mode simultaneously. The system also includes a computing device 216 that is to communicate with the ultrasound imaging device 100 through a communication channel, such as a wireless communication channel 218 as shown, although embodiments also encompass within their scope wired communication between a computing system and imaging device. The ultrasound imaging device 100 may communicate signals to the computing device 216 which may have one or more processors to process the received signals to complete formation of an image of the object. A display device 220 of the computing system 222 may then display images of the object using the signals from the computing device.

An imaging device according to some embodiments may include a portable device, and/or a handheld device that is adapted to communicate signals through a communication channel, either wirelessly (using a wireless communication protocol, such as an IEEE 802.11 or Wi-Fi protocol, a Bluetooth protocol, including Bluetooth Low Energy, a mmWave communication protocol, or any other wireless communication protocol as would be within the knowledge of a skilled person) or via a wired connection such as a cable (such as USB2, USB 3, USB 3.1, and USB-C) or such as interconnects on a microelectronic device, with the computing device. In the case of a tethered or wired, connection, the ultrasound imaging device may include a port for receiving a cable connection of a cable that is to communicate with the computing device. In the case of a wireless connection, the ultrasound imaging device 100 may include a wireless transceiver to communicate with the computing device 216.

It should be appreciated that, in various embodiments, different aspects of the disclosure may be performed in different components. For example, in one embodiment, the ultrasound imaging device may include circuitry (such as the channels) to cause ultrasound waveforms to be sent and received through its transducers, while the computing device may be adapted to control such circuitry to the generate ultrasound waveforms at the transducer elements of the ultrasound imaging device using voltage signals, and further a processing of the received ultrasonic energy.

FIG. 3 represents a view of an imaging device according to some embodiments, as will be described in further detail below.

As seen in FIG. 3, the ultrasound imaging device 300 may include a handheld casing or housing 331 where transducers 302 and associated electronics are housed. The ultrasound imaging device may also contain a battery 338 to power the electronics. FIG. 3 thus shows an embodiment of a portable imaging device capable of 2D and 3D imaging using pMUTs in a 2D array, optionally built on a silicon wafer. Such an array coupled to an application specific integrated circuit (ASIC) 106 with electronic configuration of certain parameters, enables a higher quality of image processing at a low cost than has been previously possible. Further by controlling certain parameters, for example the number of channels used, power consumption may be altered, and temperature may be changed.

FIG. 3 is a schematic diagram of an imaging device 300 with selectively adjustable features, according to some embodiments. The ultrasound imaging device 300 may be similar to imaging device 100 of FIG. 1, or to imaging device 202 of FIG. 2, by way of example only. As described above, the ultrasound imaging device may include an ultrasonic medical probe. FIG. 3 depicts transducer(s) 302 of the ultrasound imaging device 300. As described above, the transducer(s) 302 may include arrays of transducer elements (FIG. 1, 104) that are adapted to transmit and receive pressure waves (FIG. 2, 210). In some examples, the ultrasound imaging device 300 may include a coating layer 322 that serves as an impedance matching interface between the transducers 302 and the human body, or other mass or tissue through which the pressure waves (FIG. 2, 210) are transmitted. In some cases, the coating layer 322 may serve as a lens when designed with the curvature consistent with focal length desired.

The ultrasound imaging device 300 housing 331 may be embodied in any suitable form factor. In some embodiments, part of the ultrasound imaging device 300 that includes the transducers 302 may extend outward from the rest of the ultrasound imaging device 100. The ultrasound imaging device 300 may be embodied as any suitable ultrasonic medical probe, such as a convex array probe, a micro-convex array probe, a linear array probe, an endovaginal probe, endorectal probe, a surgical probe, an intraoperative probe, etc.

In some embodiments, the user may apply gel on the skin of a living body before a direct contact with the coating layer 322 so that the impedance matching at the interface between the coating layer 322 and the human body may be improved. Impedance matching reduces the loss of the pressure waves (FIG. 2, 210) at the interface and the loss of the reflected wave travelling toward the ultrasound imaging device 300 at the interface.

In some examples, the coating layer 322 may be a flat layer to maximize transmission of acoustic signals from the transducer(s) 102 to the body and vice versa. The thickness of the coating layer 322 may be a quarter wavelength of the pressure wave (FIG. 2, 210) to be generated at the transducer(s) 102.

The ultrasound imaging device 300 also includes a control circuitry 106, such as one or more processors, optionally in the form of an application-specific integrated circuit (ASIC chip or ASIC), for controlling the transducers 102. The control circuitry 106 may be coupled to the transducers 102, such as by way of bumps.

The ultrasound imaging device 300 includes sensor circuitry 335 coupled to the communication circuitry 332 and to the processor circuitry 326. The sensor circuitry 335 may include any sensor circuitry to sense at least a tap on the ultrasound imaging device housing, a tilt or orientation of the ultrasound imaging device.

The ultrasound imaging device may also include one or more processors (or processing circuitries) 326 for controlling the components of the ultrasound imaging device 300. One or more processors 326 may be configured to, in addition to control circuitry 106, at least one of control an activation of transducer elements, process signals based on reflected ultrasonic waveforms from the transducer elements or generate signals to cause generation of an image of an object being imaged by one or more processors of a computing device, such as computing device 112 of FIG. 1 or 216 of FIG. 2. One or more processors 326 may further be adapted to perform other processing functions associated with the ultrasound imaging device.

The one or more processors 326 may be embodied as any type of processors 326. For example, the one or more processors 326 may be embodied as a single or multi-core processor(s), a single or multi-socket processor, a digital signal processor, a graphics processor, a neural network compute engine, an image processor, a microcontroller, a field programmable gate array (FPGA), or other processor or processing/controlling circuit.

The ultrasound imaging device 300 may also include circuitry 328, such as Analog Front End (AFE), for processing/conditioning signals.

The analog front end 328 may be embodied as any circuit or circuits configured to interface with the control circuitry 106 and other components of the ultrasound imaging device, such as the processing circuitry 326. For example, the analog front end 328 may include, e.g., one or more digital-to-analog converters, one or more analog-to-digital converters, one or more amplifiers, etc.

The ultrasound imaging device may include a communication unit 332 for communicating data, including control signals, with an external device, such as the computing device (FIG. 2, 216), through for example a port 334 or a wireless transceiver. The ultrasound imaging device 300 may include memory 336 for storing data. The memory 336 may be embodied as any type of volatile or non-volatile memory or data storage capable of performing the functions described herein. In operation, the memory 336 may store various data and software used during operation of the ultrasound imaging device 300 such as operating systems, applications, programs, libraries, and drivers.

In some examples, the ultrasound imaging device 300 may include a battery 338 for providing electrical power to the components of the ultrasound imaging device 300. The battery 338 may also include battery charging circuits which may be wireless or wired charging circuits (not shown). The ultrasound imaging device may include a gauge that indicates a battery charge consumed and is used to configure the ultrasound imaging device to optimize power management for improved battery life. Additionally or alternatively, in some embodiments, the ultrasound imaging device may be powered by an external power source, such as by plugging the ultrasound imaging device into a wall outlet.

The sensor circuitry 335 may be coupled to housing 331 to sense an inertial change at the housing, and to cause, based on the sensed inertial change, one or more ultrasound exam functions to be executed at a computing system associated with the ultrasound imaging device. By way of example, the housing 331 may have a rigid body, and the sensor circuitry 335 may be coupled to the body of the housing such that inertial changes at the housing may be captured as sensor signals that correspond to the inertial changes.

Inertial changes may correspond of one or more taps by the guiding hand of the user on the housing of the ultrasound imaging device.

Either the sensor circuitry itself, or sensor signal processing circuitry (sensor signal processing circuitry) 337 distinct from the sensor circuitry, may be configured to use the signals based on a sensed inertial change, and correlate the signals with a tap pattern associated with an ultrasound exam function. For example, the sensor signal processing circuitry may be in the processing circuitry 326 of the ultrasound imaging device 300, or it may be distinct from it (not shown).

A tap pattern may include a permutation of one or more tap sequences. A tap sequence may include a single tap or any number of closely spaced (in time) taps. The tap pattern may include any number of such tap sequences. For example a tap pattern may include a single tap, a double tap, a triple tap, a closely spaced sequence of n taps, a permutation including any number of closely spaced taps followed by any other number of closely spaced taps (e.g. a double tap followed by a quadruple tap, a single tap followed by a double tap, etc.).

The sensor signal processing circuitry 337 may use a plurality of tap patterns and correlate each of the tap patterns with a corresponding one of a plurality of ultrasound exam functions.

Thus, the plurality of tap patterns may include a set of tap patterns that is either preconfigured to the sensor signal processing circuitry, or configurable to the sensor signal processing circuitry by a user. Different patterns of inertial change, such as a single tap, a double tap, a triple tap, any number of taps, and any permutation of tap sequences (such as, for example, a single tap followed by a double tap, a double tap followed by a quadruple tap, a single tap followed by a double tap followed by a single tap, etc.) may correspond to inertial change sensed by the sensor circuitry.

The time delta between taps may be preconfigured by way of logic within the sensor signal processing circuitry such that it can discern the numbers of taps within a given tap sequence (i.e. single tap, double tap, etc.) and the permutations of sequences of tap numbers (e.g. a permutation including a single tap sequence followed by double tap sequence, another pattern including a double tap sequence followed by a single tap sequence, etc.).

The sensor circuitry may be coupled to the housing to detect inertial change over a majority of the surface of the housing, over a bottom half of the housing, a top half of the housing, over the bottom 70% of the housing, or at any given surface area of the housing. Preferably, the sensors are coupled to the housing to detect inertial change at a bottom 70% of the housing, as this is where the user's hand is likely to be and to cause inertial change without disturbing the user's grip during an ultrasound exam.

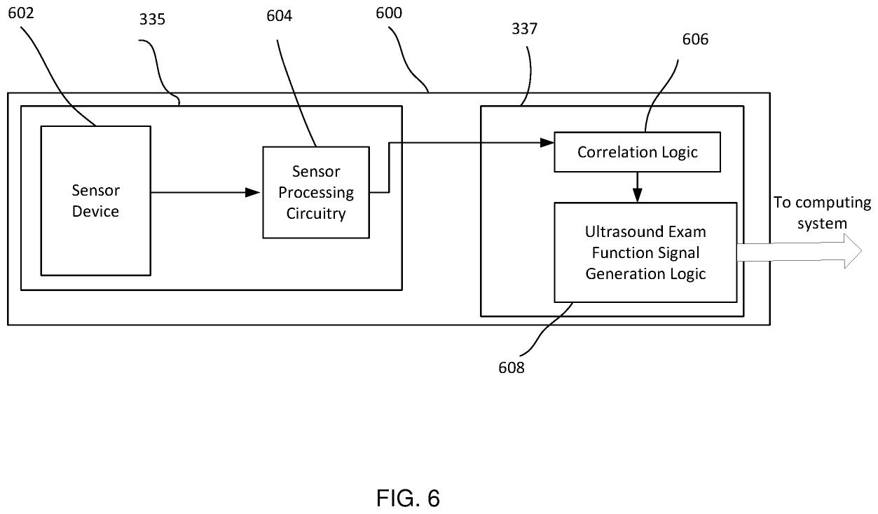

The sensor circuitry 335 may, for example, include an accelerometer. The sensor circuitry may, additionally, include a gyroscope to sense tilt or orientation of the ultrasound imaging device or its angular velocity, and/or a magnetometer to sense an ambient magnetic field of the earth to allow determination of location relative to an earth's pole. More detail regarding the sensor circuitry 335 and associated processing circuitry will be provided in the context of FIG. 6 below.

The sensor circuitry is to allow inertial change, such as taps on the housing body to be sensed, and for signals relating to the sensed inertial change to be further processed, for example to determine a correlation between the signals relating to sensed inertial change. The correlation may be performed by the sensor signal processing circuitry 337, which, in the shown embodiment, is depicted as a circuitry that is distinct from the sensor circuitry. However, embodiments are not so limited. The sensor signal processing circuitry may be within the sensor circuitry 335, within the processing circuitry 326, or within a computing system 222 that is separate from the imagine device, such as computing device 216 of the embodiment of FIG. 2.

Some embodiments advantageously allow inertial change as sensed on the housing of an imaging device to control ultrasound exam functions at a computing system, in this manner obviating the need for a user to re-adjust his/her guiding hand position on the ultrasound imaging device, in this manner doing away with the need for physical adjustments of a user's grip on the ultrasound imaging device.

Some embodiments advantageously allow a user the flexibility to hold the probe in whatever manner is comfortable to the user.

Reference is now made to FIGS. 4A and 4B, which show perspective views of a state of the art handheld ultrasonic probe 400 being held in two different manners: in FIG. 4A in a standard longitudinal grip, and in FIG. 4B, in a standard transverse grip. FIGS. 4A and 4B merely show two different types of grips for holding the shown ultrasonic probe, although many manners of holding an ultrasonic probe are possible, including an adjusted transverse grip where the probe is held and guided at top side regions thereof, and an adjusted longitudinal grip where the probe is held and guided at top front and back regions thereof. Probe 400 may be coupled to the computing system and/or to a power source by way of a wire 454 partially shown in FIGS. 4A and 4B.

Probe 400 includes a casing or housing body 440 which corresponds to the physical body of the device to be held by a user during use, such as during an ultrasonic scan or ultrasonic examination (exam). Probe 400 further includes a top half 442 and a bottom half 444. The bottom half 444 includes a probe head region 446 with a surface 448 that is to be placed in contact with the surface of the body to be scanned, such as with human skin. The top half 442 includes an actuatable button 450 that may be actuated (physically moved such as by being depressed or flipped on/off) by one or more fingers of a user, such as of the user whose hand 452 is shown in the image. The actuatable button 450 is placed in the shown example at the top region, although some handheld ultrasonic probes place the button in a middle region thereof between the top and the bottom regions. The button is typically used by a user in order to cause execution by a computing system of ultrasound exam functions, such as functions which may include, for example, freezing/unfreezing an ultrasonic image (hereinafter “image”), saving an image, taking a snapshot of an image.

During a standard ultrasound examination, according to the state of the art, with a handheld ultrasound device, a user would typically hold and guide the ultrasound imaging device in one hand (the guiding hand) and use another hand to interact with a computing system or computing device, or to guide a needle or catheter. For example, the computing system may be similar to computing system 222 and may include a mobile device. The user would therefore typically then hold the ultrasound imaging device 400 with hand 452, and utilize a finger, such as the thumb 456 to depress a physical button 450 in order to cause the computing device coupled to the probe 400 to perform ultrasound exam functions. In some state of the art probes, there is no button on the probe, and, in such instances, the user would have to use his/her guiding hand to hold and guide the probe and use his/her other hand to interact with a user interface of a computing system or computing device in order to cause execution of ultrasound exam functions.

The necessity to use the guiding hand 452 in certain cases to cause execution of ultrasound exam functions however creates a “third hand problem.” The “third hand problem” refers to the challenge of causing execution of ultrasound exam functions while one hand is either interacting with a user interface of a computing system or computing device (in a diagnostic ultrasound) or guiding a needle or catheter during a procedural ultrasound exam, and another hand is holding and guiding the handheld ultrasonic probe. In such a case, a “third hand” would theoretically be needed in order to push the button 450 when an ultrasound exam function is sought to be executed without interfering with the control exerted on the probe by the guiding hand 452. In addition, for the hand that is to interact with the computing system, the user performing the exam typically wears gloves, and may as a result not have the ability to effectively interact with the user interface of the computing system associated with the probe. For example, the user may, with gloves on his/her hands, not be able to effectively navigate the functions of the mobile device to adjust or capture and save images. Thus, in the state of the art, there is a third hand missing in order to be able to both effectively hold and guide the ultrasound imaging device such as probe 400 on the one hand, and cause execution of ultrasound exam functions at a computing system on the other hand.

Current solutions for the third hand problem other than a button as shown in the context of FIGS. 4A and 4B include foot pedals.

Some prior art solutions therefore limit the ability to effectively guide the probe as they require the user to relocate his/her finger along a height direction h of the probe to activate a button, in this way interfering with a reliable positioning (e.g. position on the skin, tilt, orientation, direction of movement, velocity) of the ultrasonic probe because of the associated movement of the guiding hand. Some other prior art solutions require the non-guiding hand to interface with a user interface of a computing system associated with the ultrasonic probe, thus limiting the ability of that non-guiding hand to perform procedures during an ultrasonic exam, such as a procedure involving the use of a needle or catheter. Some further prior art solutions present cumbersome mechanisms, such as a foot pedal, require the use of a user's body parts that are not as easily controllable as a hand, and which necessitate complicated device connections.

Reference is now made to FIGS. 5A and 5B, which show perspective views similar to those of FIGS. 4A and 4B, but according to one embodiment. A difference between probe 400 of FIGS. 4A-4B and probe 300 of FIGS. 5A-5B is that probe 300 does not include a button 450, but instead corresponds to probe 300 of FIG. 3, which includes sensor circuitry 335 described above. Probe 300 of FIG. 5 may have a computing system associated therewith, that is, a computing system in communication therewith to receive ultrasound image signals therefrom, and to display the same on a display, similar to computing system 222 of FIG. 2 by way of example.

Probe 300 may be coupled to the computing system and/or to a power source by way of a wire 554 partially shown in FIGS. 4A and 4B. The probe 300 of FIGS. 4A and 4B may further include some internal components similar to internal components shown or discussed with respect to any of the ultrasound imaging devices 100-300 described previously. Alternatively, probe 300 may include communication circuitry 332 (see FIG. 3) in order to be able to wirelessly communicate with its computing system 222 (see FIG. 2).

Probe 300 includes a casing or housing body 540 which corresponds to the physical body of the device to be held by a user during use, such as during an ultrasonic scan or ultrasonic examination (exam). Probe 300 further includes a top half 542 and a bottom half 544. The bottom half 544 includes a probe head region 546 with a surface 548 that is to be placed in contact with the surface of the body to be scanned, such as with human skin.

Because probe 300 includes sensor circuitry 335, it may allow a user, merely be using a tap pattern configured to the associated sensor signal processing circuitry 337, to be able to control ultrasound exam functions, such as those relating to imaging functions on the display of computing system 222.

Some embodiments advantageously provide an ultrasound imaging device, such as an ultrasonic probe, that includes sensor circuitry coupled to a housing thereof to sense inertial changes (hereinafter sometimes referred to as “haptic input”) at the housing, and to cause, based on the sensed inertial changes, one or more ultrasound exam functions to be executed at a computing system associated with the ultrasound imaging device, the ultrasound exam functions including functions to control an ultrasound image on a display of the computing system.

The sensor circuitry may send information based on the sensed inertial changes to a processing circuitry, the processing circuitry to determine a correlation between the sensed inertial changes and one or more ultrasound exam functions to be executed at the computing system.

The inertial change may correspond to one or more taps by the guiding hand of the user on the housing of the ultrasound imaging device. Different types of inertial change, such as a single tap, a double tap, a triple tap, any number of taps, and any combination of tap sequences (such as, for example, a single tap followed by a double tap, a double tap followed by a quadruple tap, a single tap followed by a double tap followed by a single tap, etc.) may correspond to inertial change sensed by the one or more sensor circuits. The time delta between taps, or a maximum time duration/window for a tap sequence based on the number of taps to be within that sequence, may be preconfigured, or configured by the user, by way of logic within the sensors or associated processing circuitry such that the processing circuitry can discern the numbers of taps and the combinations of tap numbers.

An inertial change may further correspond to movement of the ultrasound imaging device in the air, for example, to air drawing using the ultrasound imaging device. In such a case, the sensor circuitry may be adapted to sense motion patterns of the ultrasound imaging device in the air. Air drawing may be useful for parts of the exam where the ultrasound imaging device may not need to be on the patient's skin, such as, for example, at the end of an exam.

An inertial change may further correspond to changes in the roll, pitch, yaw, position, gravity vector, and/or linear acceleration of the ultrasound imaging device.

The sensor circuitry may be coupled to the housing to detect inertial change over a majority of the surface of the housing, over a bottom half of the housing, a top half of the housing, over the bottom 70% of the housing, over the bottom 90% of the housing, or at any given surface area of the housing. Preferably, the sensors are coupled to the housing to detect inertial change at a bottom 70% of the housing or at a bottom 50% of the housing.

Embodiments further include instances where haptic input (that is, input from a user associated with inertial changes detected by the sensor circuitry) may be combined with, or replaced with, other sensor input to cause execution of an ultrasonic exam function at an associated computing system.

By way of example, sensor circuitry 335, in addition to, or instead of, sensor circuitry to receive and decode haptic input into inertial change signals as described above, may include an audio sensor such as microphone circuitry, an eye tracking sensor such as a camera circuitry, and/or other sensors that do not involve the actuation (such as depression) of a physical button.

For example, where the sensor circuitry includes eye tracking circuitry, eye tracking input may correspond to one or more blinks by the user of the ultrasound imaging device. Different types of eye tracking input, such as a single blink, a double blink, a triple blink, any number of blinks, and any combination of blink sequences (such as, for example, a single blink followed by a double blink, a double blink followed by a quadruple blink, a single blink followed by a double blink followed by a single blink, etc.) may correspond to eye tracking input sensed by the sensor circuitry. The time delta between blinks may be preconfigured, or configured by the user, by way of logic within the sensors or associated processing circuitry such that the processing circuitry can discern the numbers of blinks and the combinations of blink numbers.

Eye tracking input may further include tracking a movement pattern of an iris of a user.

Although not shown herein, embodiments include within their scope the provision of an ultrasound imaging device that includes haptic, eye tracking and/or audio sensor circuitries alongside an actuatable button.

According to some embodiments, a sensor signal processing circuitry may thus include circuitry to process signals based on sensor input other than haptic (inertial change) sensor input, and to correlate such signals with a pattern of sensor input associated with an ultrasound exam function. Where the sensor signal processing circuitry is to process signals based on various types of sensor inputs (such as haptic and eye tracking for example), the sensor signal processing circuitry may include distinct processing circuitry components to identify the signals from various types of sensor circuitries (e.g. inertial change, eye tracking, and audio) as specific patterns of sensor input, and to generate signals based on the distinct patterns of sensor input for further processing, that is, for correlation to a subsequent sensor signal processing circuitry with an ultrasound exam function.

For voice/audio sensor input and eye tracking sensor input, the sensor circuitry configured to determine those inputs may be disposed on either the ultrasound imaging device or on the computing system or split between the ultrasound imaging device or the computing system (e.g. the eye tracking sensor circuitry could be on the ultrasound imaging device, or integrated within or attached to the display that outputs ultrasound images (where the user may frequently be looking already), and the audio sensing circuitry could be on the computing system). Alternatively, such sensor circuitry may be placed in an exam room.

Sensor signal processing circuitry may be configured to correlate any one pattern of sensor inputs with any one ultrasound exam function below.

Any one example pattern of sensor input may, by way of example only, include one or more of the following sensor inputs in any given order:
1. Haptic/inertial change input:
   a. Single tap
   b. Double tap
   c. Triple tap
   d. Quadruple tap
   e. N consecutive taps
2. Eye tracking input:
   a. Long blink
   b. Single blink
   c. Double blink
   d. Triple blink
   e. Quadruple blink
   f. M consecutive blinks
3. Voice command input:
   a. Spoken word
   b. Any sound that can be readily discernable

Thus, according to some embodiments, any one pattern of sensor input may, by way of example only, include, for example, any permutation (that is, in a given order) of sensor inputs selected from 1.a through 1.d, 2.a through 2.f and 3.a through 3.b, with an example permutation including only one of the sensor inputs above (e.g. 1.b), or a plurality of sensor inputs in a given order (e.g. 1.b followed by 2.c; or 1.b. followed by 2.c followed by 3.a; 3.a followed by another 3.a; 1.a followed by 1.b; 2.a followed by 2.f followed by 2.c followed by 1.d, etc.).

Any one pattern of sensor input as described above, according to an embodiment, may be associated with one of a set of ultrasound exam functions, either by being preconfigured to the sensor signal processing circuitry, or by being configured by the user to the sensor signal processing circuitry.

According to an embodiment, the sensor signal processing circuitry may be reconfigurable to associate different patterns of sensor input to different ultrasound exam functions at different times and/or for different users. Thus, for example, for one user, a correlation between the available patterns of sensor input may be different than for another user, and the computing system may be configured to select between the various correlations based on input from the user regarding his/her identity.

A set of ultrasound exam functions may include, for example, any of the below exam functions, where saving is performed by saving to a memory such as memory 336 or memory 251, that is, to memory that may be part of an ultrasound imaging device (such as ultrasound imaging device 300), or part of a computing system (such as imaging system 222):
a. freeze/unfreeze (freezes or unfreezes an ultrasound image of a display)
b. save (saves an ultrasound image of a display, especially after freezing)
c. snapshot (saves an ultrasound image of a display without prior freezing)
d. start/stop recording (recording is of an ultrasound video on a display, where recording is saved)
e. depth up/down (adjusts the imaging depth within a body being examined)
f. gain up/down (adjusts brightness of the image on the display)
g. activate voice commands (voice commands may be activated separately, especially after user has ensured that environment is quiet to avoid inadvertent functions being activated)
h. activate voice annotations
i. mode on/off (color Doppler (CD), motion mode (MM), pulse wave (PW))
j. increase/decrease field of view (FOV) (increases or decreases angular image corresponding to a target area being examined)
k. start 3D sweep (3D sweeps may include a sweep by transducers of the ultrasound imaging device of a volume surrounding and including the target being examined)
l. begin exam
m. signing off exam
n. end exam
o. measurements
p. tagging view
q. menu navigation (allows menu navigation among various menus and submenus of ultrasound exam functions, such as beginning exam, followed by freezing, followed by saving, etc.).
r. preset selection (allows selection of preset exam functions for example based on user, and/or based on the target being imaged, such as a kidney versus a heart, etc.)
s. annotation selection
t. worksheet selection
u. worksheet completion
v. switch to a low power standby mode
w. wake-up

Thus, according to some embodiments, any one pattern of sensor input may be associated with, and therefore correlated by the sensor signal processing circuitry, with any given one of the ultrasound exam functions a-cc noted above. According to an embodiment, ultrasound exam functions a-n may be considered “basic ultrasound exam functions.”

Table 1 provides an example of a correlation between a set of sensor circuitry input types (whether haptic/based on eye tracking and possible ultrasound exam functions). As suggested by Table 1, simpler patterns of sensor input may be associated with more common ones of the ultrasound exam functions, such as those outlined in items a. through f. of the set of imaging functions above.

TABLE 1

| Sensor Circuitry Input | Ultrasound Exam | Pattern of |
|---|---|---|
| Types | Function | Sensor Input |
| haptic/inertial motion | freeze/unfreeze | single tap |
| haptic/inertial motion | save | double tap |
| haptic/inertial motion | snapshot | triple tap |
| haptic/inertial motion | start/stop recording | double tap, single |
| | | tap |
| haptic/inertial motion | depth up/down | single tap, double |
| | | tap |
| haptic/inertial motion | gain up/down | air drawing |
| eye tracking | activate voice commands | long blink |
| eye tracking | activate voice | Double blink |
| | annotations | |
| eye tracking | mode on/off (CD, MM, | right wink |
| | PW) | |
| eye tracking | increase/decrease FOV | left wink |
| eye tracking | start 3D sweep | double right wink |
| air Drawing & eye tracking | begin exam | double left wink |
| air Drawing & eye tracking | signing off exam | dwell |
| haptic, air drawing, eye | end exam | nod up |
| tracking | | |
| eye tracking | measurements | nod down |
| eye tracking | tagging view | nod left |
| eye tracking | menu navigation | nod right |
| eye tracking | preset selection | |
| eye tracking | annotation selection | |
| eye tracking | worksheet selection | |
| eye tracking | worksheet completion | |

Reference is now made to FIG. 6, which corresponds to a schematic illustration of an embodiment of sensor circuitry 335 and of sensor signal processing circuitry 337 according to an embodiment where both components are within a single package 600, such as a chip, a system on a chip, or a microelectronic package, to name a few In the shown embodiment, sensor circuitry includes a sensor device 602, and a sensor processing circuitry 604.

The sensor device 602 may include, by way of example, an accelerometer to sense haptic/inertial change input to the housing of the ultrasound imaging device, such as one or more taps, and air drawing gestures. The sensor device may further include a gyroscope to determine a positioning of the ultrasound imaging device, such as for example its tilt angle with respect to a surface of the skin of a patient being examined or its angular velocity, and/or a magnetometer/compass in order to measure the earth's ambient magnetic field, and to allow any adjustments needed with respect to the data from the gyroscope. The sensor device may, in addition, to the accelerometer, further include an audio sensor, such as a microphone to sense audio input, or an eye tracking sensor, such as a camera. The sensor device is to detect inertial change/one or more of ultrasound imaging device orientation/tilt/angular velocity, an audio input and eye tracking input, and generate sensor data therefrom. Each type of sensor device (e.g. accelerometer, gyroscope, magnetometer, microphone, camera) may generate its own sensor data/sensor raw data and send it for further processing to sensor processing circuitry 604. For example, the accelerometer may generate raw data corresponding to a waveform based on inertial change. The magnetometer may generate raw data corresponding to a value for earth's ambient magnetic field. The gyroscope may generate raw data corresponding to angular tilt of the ultrasound imaging device.

The sensor raw data from each sensor device may be processed by the sensor processing circuitry 604, which may, according to one embodiment, include logic to fuse sensor data relating to haptic input/inertial changes. For example, the raw data from the magnetometer may be used in conjunction with that from the accelerometer and/or gyroscope to compensate for any errors with respect to the raw data from the accelerometer and/or gyroscope. In addition, the raw data from the gyroscope with respect to angular tilt may be used in conjunction with the raw data from the accelerometer in order to determine whether the ultrasound imaging device is in a desired position with respect to a target to be imaged prior to ultrasound exam functions being executed on the target's ultrasound image. By way of example, if the ultrasound imaging device is to be held still during an ultrasound exam function, such as, for example, freezing/unfreezing, taking a snapshot, or performing a 3D sweep, the sensor processing circuitry, by virtue of its logic to fuse sensor data, may cause feedback to a user regarding the ultrasound imaging device not yet being stationary. In this manner, the user may hold the ultrasound imaging device in a stationary position as a result of the feedback in order to cause execution of ultrasound exam functions that are dependent on an inertial status of the ultrasound imaging device as being stationary. Thus, according to one embodiment, the fusion algorithm may use the raw data from the gyroscope and from the accelerometer to determine whether the inertial status of the ultrasound imaging device is consistent with the ultrasound exam function being sought to be implemented (as explained above), and, if an inconsistency is found, to cause feedback to be provided to the user (either through the ultrasound imaging device itself, or by way of the computing system associated with the ultrasound imaging device) in order to adjust the inertial status of the ultrasound imaging device to one that is consistent with the ultrasound exam function sought to be implemented. By way of example, the fusion algorithm may use the raw data from the gyroscope and the accelerometer to determine whether the inertial status of the ultrasound imaging device is consistent with the preset status of the ultrasound exam to be or being performed. For example, if the inertial status of the ultrasound imaging device is inconsistent with the preset status of the ultrasound exam, the feedback to the user from either the ultrasound imaging device or the computing system may include information to allow the user to adjust the inertial status to be within range of inertial statuses consistent with the chosen presets, or to change the presets to be consistent with the existing inertial status.

Ultrasound presets include many of the common ultrasound imaging parameters, such as dynamic range, depth. focal zone, persistence, automatic gain control (e.g. auto and tissue equalization), compounding for both spatial and frequency, sine functions, line density, tint maps, middle frequency, measurements, annotations, settings for tissue border delineation, to name a few. Presets may also include an M-Mode, Doppler, color Doppler, access to continuous wave Doppler, 3D/4D and even elastography and contrast parameters. Each parameter can be changed independently within a preset to improve the images.

The inertial status may include information based on whether the ultrasound imaging device is still/stationary, the ultrasound imaging device's tilt in relation to the skin surface of the patient on which the ultrasound imaging device is placed, and/or the mobility state of the ultrasound imaging device (whether it is moving, how fast, in what direction).

The feedback may be haptic, by way of audio, and/or by way of a visual display, such as on the display of the computing system, or by way of a light source that is part of the ultrasound imaging device (e.g. flashing lights, red light, green light, etc.).

The sensor processing circuitry 604 may use quaternion calculations to obtain information on ultrasound imaging device orientation, tilt, angular velocity, position change.

The sensor processing circuitry 604 may further use the raw sensor data, and generate signals based on the same that correlate with information regarding the pattern of sensor input detected, for example any pattern of sensor input as described above. As previously noted, for example, any one pattern of sensor input may include any permutation of sensor inputs selected from 1.a through 1.d, 2.a through 2.f and 3.a through 3.b above, with an example permutation including only one of the sensor inputs above (e.g. 1.b), or a plurality of sensor inputs in a given order (e.g. 1.b followed by 2.c; or 1.b. followed by 2.c followed by 3.a; 3.a followed by another 3.a; 1.a followed by 1.b; 2.a followed by 2.f followed by 2.c followed by 1.d, etc.). The sensor processing circuitry may generate signals based on the detected pattern of sensor input. Alternatively, the ultrasound imaging device may simply obtain the raw sensor data from its sensor device(s) and send the raw sensor data to its associated computing system, such as computing system 222 of FIG. 2, in which case the sensor processing circuitry 604 would be in whole or in part housed within the computing system rather than within the ultrasound imaging device.

For example, for tap detection, the sensor processing circuitry may determine, from a processing of the raw accelerometer data, whether any sharp pulses (e.g. about 150 ms to about 375 ms in length with a jump above about 0.125 g in acceleration) and extracts the number of such sharp pulses to detect taps, the time duration between the taps, etc. As noted previously, a time delta between taps, or a maximum time delta for the detection of a given number of taps, may be preconfigured to the sensor processing circuitry 604, or configurable to the same by a user. The sensor processing circuitry may be configured with a maximum time window for N taps, a minimum time window for one tap, a time delta (duration) between sequences of N taps each, an amplitude threshold beyond which a change in acceleration qualifies as a tap gesture, to name a few.

As further seen in FIG. 6, the signals based on the detected pattern of sensor input may be sent by the sensor processing circuitry 604 to the sensor signal processing circuitry 337. In the shown example, the signals based on the detected pattern of sensor input may be processed using correlation logic within the sensor signal processing circuitry to correlate the pattern of sensor input with one of a set of ultrasound exam functions, such as, for example, those listed under a.-cc. above. In order to perform the above, the correlation logic may cause the sensor signal processing circuitry to access data relating to the set of configured patterns of sensor inputs and their corresponding set of ultrasound exam functions, such data for example being comparable with that presented in Table 1 above. Based on the correlation, an ultrasound exam function signal generation logic 608 may cause the sensor signal processing circuitry 337 to generate signals to cause execution, by the computing system, of the ultrasound exam function with which a pattern of sensor input was correlated. The signals to cause execution may be sent by the sensor signal processing circuitry 337 to the ultrasound imaging device's communication circuitry for communication to the computing system, for example to communication circuitry 332. For a mobile ultrasound imaging device, the signals to cause execution may be communicated by way of a wireless air medium or by way of wires to the computing system.

According to an embodiment, an inertial motion sensor device, such as an accelerometer, and one or more of a gyroscope and a magnetometer, may be coupled to the housing to detect inertial change over a majority of the surface of the housing. As noted previously, the inertial motion sensor may be placed to sense inertial change over a bottom half of the surface of the surface of the housing, a top half of the surface of the housing, over the bottom 70% of the surface of the housing, or at any given surface area of the housing. Preferably, the sensors are coupled to the housing to detect inertial change at a bottom 70% of the housing, as this is where the user's hand is likely to be and to cause inertial change without disturbing the user's grip during an ultrasound exam. According to an embodiment, the sensor circuitry may be configured to change a sensitivity of the inertial motion sensor device based on a location on a surface area of the housing where an inertial change, such as a tap, is detectable by the sensor device. Preferably, a sensitivity of the inertial motion sensor may be higher for taps on a lower surface area of the ultrasound imaging device housing than on a surface area of the ultrasound imaging device housing above that lower surface.

According to an embodiment, the sensor signal processing circuitry 337 may further correlate a pattern of sensor input with for example detection of ultrasound imaging device user-related events, such as whether the probe has been picked up by a user, whether it is in rest (not being used in an exam), or whether it has been dropped. The user related events, once detected by the signal processing circuitry 337 by the correlation, may in turn generate signals to the ultrasound imaging device or to the computing system regarding power settings of at least one of the ultrasound imaging device or the computing system. For example, upon detection of the ultrasound imaging device being picked up, the power supply to the ultrasound imaging device and/or computing system may be increased by virtue of a corresponding signal from the sensor signal processing circuitry. Upon detection of the ultrasound imaging device being dropped or being in rest, the power supply to the ultrasound imaging device and/or computing system may be decreased by virtue of a corresponding signal from the sensor signal processing circuitry. Upon detection of a drop, the signal from the sensor signal processing circuitry may cause indication of a warranty event.

By way of example, sensor circuitry 335, in addition to, or instead of, sensor circuitry to receive and decode haptic input into inertial change signals as described above, may include an audio sensor such as microphone circuitry, an eye tracking sensor such as a camera circuitry, and/or other sensors that do not involve the actuation (such as depression) of a physical button.

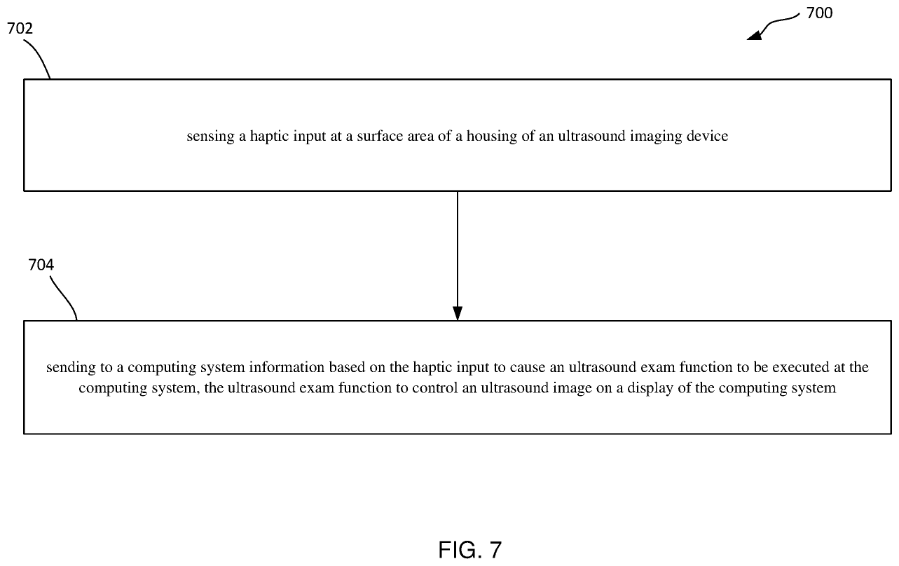

FIG. 7 shows a method 700 to be performed at an ultrasound imaging device according to one embodiment. The method includes, at operation 702, sensing a haptic input at a surface area of a housing of the ultrasound imaging device; and at operation 704, sending to a computing system information based on the haptic input to cause an ultrasound exam function to be executed at the computing system, the ultrasound exam function to control an ultrasound image on a display of the computing system.

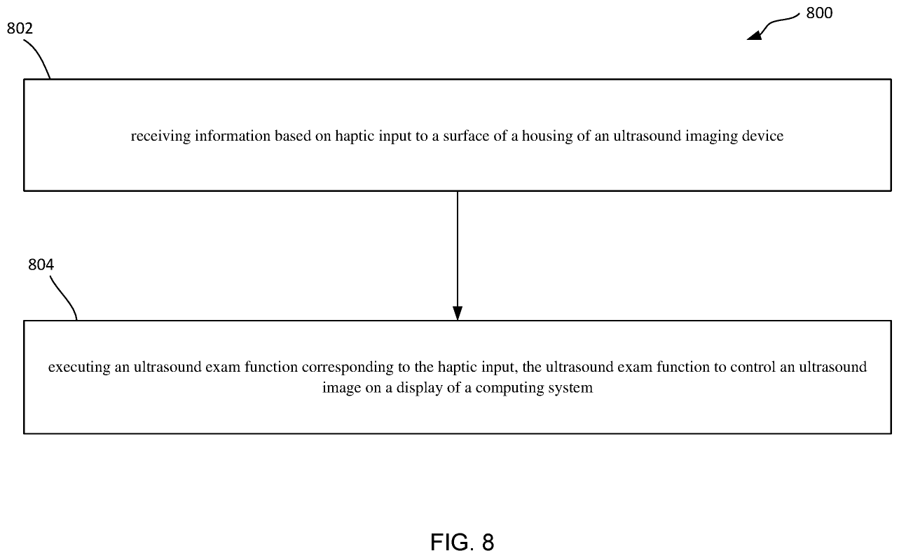

FIG. 8 shows a method 800 according to another embodiment. Method 800 includes, at operation 802, receiving information based on haptic input to a surface of a housing of an ultrasound imaging device; and, at operation 804, based on the information, executing an ultrasound exam function corresponding to the haptic input, the ultrasound exam function to control an ultrasound image on a display of a computing system.

Some embodiments advantageously allow inertial change as sensed on the housing of an imaging device to control ultrasound exam functions at a computing system, in this manner obviating the need for a user to re-adjust his/her guiding hand position on the ultrasound imaging device, in this manner doing away with the need for physical adjustments of a user's grip on the ultrasound imaging device.

Some embodiments advantageously allow a user the flexibility to hold the probe in whatever manner is comfortable.

There are several benefits associated with the use of inertial change to relay feedback to the external display. Inertial change allows the user to keep his/her hand placement/grip steady on the probe without having to make any major adjustments that could disrupt the ultrasound image or procedural exam. This also allows the user to have flexibility in his/her hand placement on the probe. No longer would a user need to conform to an uncomfortable grip on the probe to utilize physical buttons. Since inertial change is sensed internally within the probe, the probe can also be built without any external buttons that often can raise infection control concerns due to excess gel or fluids being caught in the crevasses. Physical buttons overtime can also lose sensitivity, and some are difficult to operate.

In one example, the computing system, such as computing system 222 of FIG. 2, may include a host processor device coupled to the computing device; a display communicatively coupled to a host processor; a network interface communicatively coupled to the host processor; or a battery to power the system.

The flow described in FIGS. 7 and 8 are merely representative of operations that may occur in particular embodiments. In other embodiments, additional operations may be performed by the components of the systems shown in FIGS. 1-3, 5 and 6. Various embodiments of the present disclosure contemplate any suitable mechanisms for accomplishing the functions described herein. Some of the operations illustrated in FIGS. 7 and 8 may be repeated, combined, modified, or deleted where appropriate. Additionally, operations may be performed in any suitable order without departing from the scope of particular embodiments.

A design may go through various stages, from creation to simulation to fabrication. Data representing a design may represent the design in a number of manners. First, as is useful in simulations, the hardware may be represented using a hardware description language (HDL) or another functional description language. Additionally, a circuit level model with logic and/or transistor gates may be produced at some stages of the design process. Furthermore, most designs, at some stage, reach a level of data representing the physical placement of various devices in the hardware model. In some implementations, such data may be stored in a database file format such as Graphic Data System II (GDS II), Open Artwork System Interchange Standard (OASIS), or similar format.

In some implementations, software based hardware models, and HDL and other functional description language objects can include register transfer language (RTL) files, among other examples. Such objects can be machine-parsable such that a design tool can accept the HDL object (or model), parse the HDL object for attributes of the described hardware, and determine a physical circuit and/or on-chip layout from the object. The output of the design tool can be used to manufacture the physical device. For instance, a design tool can determine configurations of various hardware and/or firmware elements from the HDL object, such as bus widths, registers (including sizes and types), memory blocks, physical link paths, fabric topologies, among other attributes that would be implemented in order to realize the system modeled in the HDL object. Design tools can include tools for determining the topology and fabric configurations of system on chip (SoC) and other hardware device. In some instances, the HDL object can be used as the basis for developing models and design files that can be used by manufacturing equipment to manufacture the described hardware. Indeed, an HDL object itself can be provided as an input to manufacturing system software to cause the described hardware.

In any representation of the design, the data may be stored in any form of a machine readable medium. A memory or a magnetic or optical storage such as a disc may be the machine readable medium to store information transmitted via optical or electrical wave modulated or otherwise generated to transmit such information. When an electrical carrier wave indicating or carrying the code or design is transmitted, to the extent that copying, buffering, or re-transmission of the electrical signal is performed, a new copy is made. Thus, a communication provider or a network provider may store on a tangible, machine-readable medium, at least temporarily, an article, such as information encoded into a carrier wave, embodying techniques of embodiments of the present disclosure.

In various embodiments, a medium storing a representation of the design may be provided to a manufacturing system (e.g., a semiconductor manufacturing system capable of manufacturing an integrated circuit and/or related components). The design representation may instruct the system to manufacture a device capable of performing any combination of the functions described above. For example, the design representation may instruct the system regarding which components to manufacture, how the components should be coupled together, where the components should be placed on the device, and/or regarding other suitable specifications regarding the device to be manufactured.

“Circuitry” as used herein may refer to any combination of hardware with software, and/or firmware. As an example, a circuitry includes hardware, such as a micro-controller, associated with a non-transitory medium to store code adapted to be executed by the micro-controller. Therefore, reference to a circuitry, in one embodiment, refers to the hardware, which is specifically configured to recognize and/or execute the code to be held on a non-transitory medium. Furthermore, in another embodiment, use of a circuitry refers to the non-transitory medium including the code, which is specifically adapted to be executed by the microcontroller to perform predetermined operations. And as can be inferred, in yet another embodiment, the term circuitry (in this example) may refer to the combination of the microcontroller and the non-transitory medium. Often circuitry boundaries that are illustrated as separate commonly vary and potentially overlap. For example, a first and a second circuitry may share hardware, software, firmware, or a combination thereof, while potentially retaining some independent hardware, software, or firmware. In one embodiment, use of the term logic includes hardware, such as transistors, registers, or other hardware, such as programmable logic devices.

Logic may be used to implement any of the flows described or functionality of the various components described herein. “Logic” may refer to hardware, firmware, software and/or combinations of each to perform one or more functions. In various embodiments, logic may include a microprocessor or other processing element operable to execute software instructions, discrete logic such as an application-specific integrated circuit (ASIC), a programmed logic device such as a field programmable gate array (FPGA), a storage device containing instructions, combinations of logic devices (e.g., as would be found on a printed circuit board), or other suitable hardware and/or software. Logic may include one or more gates or other circuit components. In some embodiments, logic may also be fully embodied as software. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in storage devices.

Use of the phrase ‘to’ or ‘configured to,’ in one embodiment, refers to arranging, putting together, manufacturing, offering to sell, importing, and/or designing an apparatus, hardware, logic, or element to perform a designated or determined task. In this example, an apparatus or element thereof that is not operating is still ‘configured to’ perform a designated task if it is designed, coupled, and/or interconnected to perform said designated task. As a purely illustrative example, a logic gate may provide a 0 or a 1 during operation. But a logic gate ‘configured to’ provide an enable signal to a clock does not include every potential logic gate that may provide a 1 or 0. Instead, the logic gate is one coupled in some manner that during operation the 1 or 0 output is to enable the clock. Note once again that use of the term ‘configured to’ does not require operation, but instead focuses on the latent state of an apparatus, hardware, and/or element, wherein the latent state the apparatus, hardware, and/or element is designed to perform a particular task when the apparatus, hardware, and/or element is operating.

Furthermore, use of the phrases ‘capable of/to,’ and or ‘operable to,’ in one embodiment, refers to some apparatus, logic, hardware, and/or element designed in such a way to enable use of the apparatus, logic, hardware, and/or element in a specified manner. Note as above that use of to, capable to, or operable to, in one embodiment, refers to the latent state of an apparatus, logic, hardware, and/or element, where the apparatus, logic, hardware, and/or element is not operating but is designed in such a manner to enable use of an apparatus in a specified manner.

The embodiments of methods, hardware, software, firmware, or code set forth above may be implemented via instructions or code stored on a machine-accessible, machine readable, computer accessible, or computer readable medium which are executable by a processing element. A tangible non-transitory machine-accessible/readable medium includes any mechanism that provides (i.e., stores and/or transmits) information in a form readable by a machine, such as a computer or electronic system. For example, a non-transitory machine-accessible medium includes random-access memory (RAM), such as static RAM (SRAM) or dynamic RAM (DRAM); ROM; magnetic or optical storage medium; flash storage devices; electrical storage devices; optical storage devices; acoustical storage devices; other form of storage devices for holding information received from transitory (propagated) signals (e.g., carrier waves, infrared signals, digital signals); etc., which are to be distinguished from the non-transitory mediums that may receive information therefrom.

Instructions used to program logic to perform embodiments of the disclosure may be stored within a memory in the system, such as DRAM, cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, Compact Disc, Read-Only Memory (CD-ROMs), and magneto-optical disks, Read-Only Memory (ROMs), Random Access Memory (RAM), Erasable Programmable Read-Only Memory (EPROM), Electrically Erasable Programmable Read-Only Memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the computer-readable medium includes any type of tangible machine-readable medium suitable for storing or transmitting electronic instructions or information in a form readable by a machine (e.g., a computer).

Some example embodiments will now be described below.

### EXAMPLES

Illustrative examples of the technologies disclosed herein are provided below. An embodiment of the technologies may include any one or more, and any combination of, the examples described below.

Example 1 includes an ultrasound imaging device including a sensor circuitry and a housing, the sensor circuitry disposed in the housing and coupled thereto to: sense a haptic input at a surface area of the housing; and send to a computing system information based on the haptic input to cause an ultrasound exam function to be executed at the computing system, the ultrasound exam function to control an ultrasound image on a display of the computing system.

Example 2 includes the subject matter of Example 1, wherein the sensor circuitry is further to send information based on the haptic input to a sensor signal processing circuitry, the sensor signal processing circuitry to determine a correlation between the sensed haptic input and one or more ultrasound exam functions to be executed at the computing system.

Example 3 includes the subject matter of Example 1, wherein the sensor circuitry includes an accelerometer.

Example 4 includes the subject matter of Example 3, wherein the sensor circuitry further includes a gyroscope.

Example 5 includes the subject matter of Example 4, wherein the sensor circuitry includes a sensor device and a sensor processing circuitry coupled to the sensor device, the sensor device including the accelerometer and the gyroscope, and the sensor processing circuitry to fuse signals corresponding to raw accelerometer data from the accelerometer with signals correspond to raw gyroscope data from the gyroscope by processing the raw accelerometer data and the raw gyroscope data to generate signals therefrom corresponding to an inertial status of the ultrasound imaging device.

Example 6 includes the subject matter of Example 5, wherein the inertial status of the ultrasound imaging device includes information based on at least one of whether the ultrasound imaging device is stationary, an angular tilt of the ultrasound imaging device with respect to a skin surface of a body being examined, an angular velocity of the ultrasound imaging device with respect to the skin, a position of the ultrasound imaging device on the skin, or a linear velocity of the ultrasound imaging device.

Example 7 includes the subject matter of Example 5, the ultrasound imaging device to receive signals based on the inertial status and to communicate feedback to a user of the ultrasound imaging device derived from the signals based on the inertial status, the feedback corresponding to an adjustment of an ultrasound examination by the user.

Example 8 includes the subject matter of Example 1, wherein the haptic input includes one or more taps on a surface of the housing.

Example 9 includes the subject matter of Example 1, wherein the haptic input includes aerial motion of the ultrasound imaging device.

Example 10 includes the subject matter of Example 1, the sensor circuitry to sense sensor input corresponding to a plurality of patterns of sensor input, each pattern of sensor input including, in a predetermined order, one or more of: the haptic input, an eye tracking input or a voice command input, each pattern of sensor input associated with a corresponding one of a plurality of ultrasound exam functions.

Example 11 includes the subject matter of Example 10, wherein the ultrasound exam functions include at least one of freezing and unfreezing the ultrasound image, saving the ultrasound image, taking a snapshot of the ultrasound image, starting and stopping a recording of ultrasound video, adjusting a depth of the ultrasound image or adjusting a gain of the ultrasound image.

Example 12 includes the subject matter of Example 10, the sensor circuitry to further determine a pattern of the plurality of patterns of sensor input, wherein the information based on the haptic input corresponds to information based on the pattern.

Example 13 includes the subject matter of Example 10, wherein the haptic input includes any one of a plurality of permutations of one or more tap sequences, a tap sequence including a single tap or any number of taps within a predetermined tap sequence time window representing a maximum time duration configured to the sensor circuitry for a tap sequence to be sensed.

Example 14 includes the subject matter of Example 10, wherein the eye tracking input includes an eye blink sequence including a single eye blink or any number of eye blinks within a predetermined eye blink time window representing a maximum time duration configured to the sensor circuitry for an eye blink sequence to be sensed.

Example 15 includes the subject matter of Example 10, further including a sensor signal processing circuitry coupled to the sensor circuitry, the sensor signal processing circuitry to determine a pattern of the plurality of patterns of sensor input, wherein the information based on the haptic input corresponds to information based on the pattern.

Example 16 includes the subject matter of Example 15, the sensor signal processing circuitry to further: perform a correlation of the pattern with its corresponding one of the plurality of ultrasound exam functions; derive from the correlation the information based on the pattern; and send the information based on the pattern to the computing system.

Example 17 includes the subject matter of Example 16, further including a memory coupled to the sensor signal processing circuitry, the memory to store information on a correlation between each pattern of sensor input of the plurality of patterns of sensor input, and corresponding ones of the plurality of ultrasound exam functions.

Example 18 includes the subject matter of Example 17, wherein information on the correlation is configurable by a user of the ultrasound imaging device.

Example 19 includes the subject matter of Example 1, further including a button on the housing, the button to be physically moved by a user to generate signals to cause one or more ultrasonic exam functions to be performed at the computing system.

Example 20 includes the subject matter of Example 1, wherein the surface area of the housing includes a bottom 70% of the housing.

Example 21 includes the subject matter of Example 1, further including a wireless transceiver to wirelessly communicate the information to the computing system.

Example 22 includes a method to be performed at an ultrasound imaging device, the method including: sensing a haptic input at a surface area of a housing of the ultrasound imaging device; and sending to a computing system information based on the haptic input to cause an ultrasound exam function to be executed at the computing system, the ultrasound exam function to control an ultrasound image on a display of the computing system.

Example 23 includes the subject matter of Example 22, further including sending information based on the haptic input to a sensor signal processing circuitry, the sensor signal processing circuitry to determine a correlation between the sensed haptic input and one or more ultrasound exam functions to be executed at the computing system.

Example 24 includes the subject matter of Example 22, wherein sensing a haptic input includes using an accelerometer.

Example 25 includes the subject matter of Example 24, wherein sensing a haptic input includes using a gyroscope.

Example 26 includes the subject matter of Example 25, further including fusing signals corresponding to raw accelerometer data from the accelerometer with signals correspond to raw gyroscope data from the gyroscope by processing the raw accelerometer data and the raw gyroscope data to generate signals therefrom corresponding to an inertial status of the ultrasound imaging device.

Example 27 includes the subject matter of Example 26, wherein the inertial status of the ultrasound imaging device includes information based on at least one of whether the ultrasound imaging device is stationary, an angular tilt of the ultrasound imaging device with respect to a skin surface of a body being examined, an angular velocity of the ultrasound imaging device with respect to the skin, a position of the ultrasound imaging device on the skin, or a linear velocity of the ultrasound imaging device.

Example 28 includes the subject matter of Example 26, further including receiving signals based on the inertial status and communicating feedback to a user of the ultrasound imaging device derived from the signals based on the inertial status, the feedback corresponding to an adjustment of an ultrasound examination by the user.

Example 29 includes the subject matter of Example 22, wherein the haptic input includes one or more taps on a surface of the housing.

Example 30 includes the subject matter of Example 22, wherein the haptic input includes aerial motion of the ultrasound imaging device.

Example 31 includes the subject matter of Example 22, further including sensing sensor input corresponding to a plurality of patterns of sensor input, each pattern of sensor input including, in a predetermined order, one or more of: the haptic input, an eye tracking input or a voice command input, each pattern of sensor input associated with a corresponding one of a plurality of ultrasound exam functions.

Example 32 includes the subject matter of Example 31, wherein the ultrasound exam functions include at least one of freezing and unfreezing the ultrasound image, saving the ultrasound image, taking a snapshot of the ultrasound image, starting and stopping a recording of ultrasound video, adjusting a depth of the ultrasound image or adjusting a gain of the ultrasound image.

Example 33 includes the subject matter of Example 31, further including determining a pattern of the plurality of patterns of sensor input, wherein the information based on the haptic input corresponds to information based on the pattern.

Example 34 includes the subject matter of Example 31, wherein the haptic input includes any one of a plurality of permutations of one or more tap sequences, a tap sequence including a single tap or any number of taps within a predetermined tap sequence time window representing a maximum time duration configured to the sensor circuitry for a tap sequence to be sensed.

Example 35 includes the subject matter of Example 31, wherein the eye tracking input includes an eye blink sequence including a single eye blink or any number of eye blinks within a predetermined eye blink time window representing a maximum time duration configured to the sensor circuitry for an eye blink sequence to be sensed.

Example 36 includes the subject matter of Example 31, further including determining a pattern of the plurality of patterns of sensor input, wherein the information based on the haptic input corresponds to information based on the pattern.

Example 37 includes the subject matter of Example 36, further including: performing a correlation of the pattern with its corresponding one of the plurality of ultrasound exam functions; deriving from the correlation the information based on the pattern; and
sending the information based on the pattern to the computing system.

Example 38 includes the subject matter of Example 37, further including storing information on a correlation between each pattern of sensor input of the plurality of patterns of sensor input, and corresponding ones of the plurality of ultrasound exam functions.

Example 39 includes the subject matter of Example 22, further including wirelessly communicating the information to the computing system.

Example 40 includes an apparatus including a memory storing instructions, and a sensor signal processing circuitry coupled to the memory to execute the instructions to: receive information based on haptic input to a surface of a housing of an ultrasound imaging device; and

based on the information, execute an ultrasound exam function corresponding to the haptic input, the ultrasound exam function to control an ultrasound image on a display of a computing system.

Example 41 includes the subject matter of Example 40, the sensor signal processing circuitry to determine a correlation between the sensed haptic input and the ultrasound exam function, and to execute the ultrasound exam function based on the correlation.

Example 42 includes the subject matter of Example 40, wherein the information includes raw accelerometer data.

Example 43 includes the subject matter of Example 42, wherein the information further includes raw gyroscope data.

Example 44 includes the subject matter of Example 43, wherein the information further includes raw magnetometer data.

Example 45 includes the subject matter of Example 44, the sensor signal processing circuitry to fuse the accelerometer data, the gyroscope data and the magnetometer data to generate signals therefrom corresponding to an inertial status of the ultrasound imaging device, and to send to the ultrasound imaging device information based on the inertial status.

Example 46 includes the subject matter of Example 45, wherein the inertial status of the ultrasound imaging device includes information based on at least one of whether the ultrasound imaging device is stationary, an angular tilt of the ultrasound imaging device with respect to a skin surface of a body being examined, an angular velocity of the ultrasound imaging device with respect to the skin, a position of the ultrasound imaging device on the skin, or a linear velocity of the ultrasound imaging device.

Example 47 includes the subject matter of Example 46, the sensor signal processing circuitry to cause communication of feedback to a user of the ultrasound imaging device, the feedback derived from the inertial status, the feedback corresponding to an adjustment of an ultrasound examination by the user.

Example 48 includes the subject matter of Example 40, wherein the haptic input includes one or more taps on a surface of the housing.

Example 49 includes the subject matter of Example 40, wherein the haptic input includes aerial motion of the ultrasound imaging device.

Example 50 includes the subject matter of Example 40, the sensor signal processing circuitry to determine a plurality of patterns of sensor input, each pattern of sensor input including, in a predetermined order, one or more of: the haptic input, an eye tracking input or a voice command input, each pattern of sensor input associated with a corresponding one of a plurality of ultrasound exam functions.

Example 51 includes the subject matter of Example 50, wherein the ultrasound exam functions include at least one of freezing and unfreezing the ultrasound image, saving the ultrasound image, taking a snapshot of the ultrasound image, starting and stopping a recording of ultrasound video, adjusting a depth of the ultrasound image or adjusting a gain of the ultrasound image.

Example 52 includes the subject matter of Example 50, wherein the haptic input includes any one of a plurality of permutations of one or more tap sequences, a tap sequence including a single tap or any number of taps within a predetermined tap sequence time window representing a maximum time duration configured to the sensor circuitry for a tap sequence to be sensed.

Example 53 includes the subject matter of Example 50, wherein the eye tracking input includes an eye blink sequence including a single eye blink or any number of eye blinks within a predetermined eye blink time window representing a maximum time duration configured to the sensor circuitry for an eye blink sequence to be sensed.

Example 54 includes the subject matter of Example 50, the sensor signal processing circuitry to determine a pattern of the plurality of patterns of sensor input, wherein the information based on the haptic input corresponds to information based on the pattern.

Example 55 includes the subject matter of Example 54, the sensor signal processing circuitry to further: perform a correlation of the pattern with its corresponding one of the plurality of ultrasound exam functions; derive from the correlation the information based on the pattern; and send the information based on the pattern to the computing system.

Example 56 includes the subject matter of Example 55, the memory to store information on a correlation between each pattern of sensor input of the plurality of patterns of sensor input, and corresponding ones of the plurality of ultrasound exam functions.

Example 57 includes the subject matter of Example 56, wherein information on the correlation is configurable by a user of the apparatus.

Example 58 includes the subject matter of Example 40, further including a wireless transceiver.

Example 59 includes a method including: receiving information based on haptic input to a surface of a housing of an ultrasound imaging device; and based on the information, executing an ultrasound exam function corresponding to the haptic input, the ultrasound exam function to control an ultrasound image on a display of a computing system.

Example 60 includes the subject matter of Example 59, further including determining a correlation between the sensed haptic input and the ultrasound exam function, and to execute the ultrasound exam function based on the correlation.

Example 61 includes the subject matter of Example 59, wherein the information includes raw accelerometer data.

Example 62 includes the subject matter of Example 61, wherein the information further includes raw gyroscope data.

Example 63 includes the subject matter of Example 62, wherein the information further includes raw magnetometer data.

Example 64 includes the subject matter of Example 63, further including fusing the accelerometer data, the gyroscope data and the magnetometer data to generate signals therefrom corresponding to an inertial status of the ultrasound imaging device, and to send to the ultrasound imaging device information based on the inertial status.

Example 65 includes the subject matter of Example 64, wherein the inertial status of the ultrasound imaging device includes information based on at least one of whether the ultrasound imaging device is stationary, an angular tilt of the ultrasound imaging device with respect to a skin surface of a body being examined, an angular velocity of the ultrasound imaging device with respect to the skin, a position of the ultrasound imaging device on the skin, or a linear velocity of the ultrasound imaging device.

Example 66 includes the subject matter of Example 65, further including causing communication of feedback to a user of the ultrasound imaging device, the feedback derived from the inertial status, the feedback corresponding to an adjustment of an ultrasound examination by the user.

Example 67 includes the subject matter of Example 59, wherein the haptic input includes one or more taps on a surface of the housing.

Example 68 includes the subject matter of Example 59, wherein the haptic input includes aerial motion of the ultrasound imaging device.

Example 69 includes the subject matter of Example 59, further including determining a plurality of patterns of sensor input, each pattern of sensor input including, in a predetermined order, one or more of: the haptic input, an eye tracking input or a voice command input, each pattern of sensor input associated with a corresponding one of a plurality of ultrasound exam functions.

Example 70 includes the subject matter of Example 69, wherein the ultrasound exam functions include at least one of freezing and unfreezing the ultrasound image, saving the ultrasound image, taking a snapshot of the ultrasound image, starting and stopping a recording of ultrasound video, adjusting a depth of the ultrasound image or adjusting a gain of the ultrasound image.

Example 71 includes the subject matter of Example 69, wherein the haptic input includes any one of a plurality of permutations of one or more tap sequences, a tap sequence including a single tap or any number of taps within a predetermined tap sequence time window representing a maximum time duration configured to the sensor circuitry for a tap sequence to be sensed.

Example 72 includes the subject matter of Example 69, wherein the eye tracking input includes an eye blink sequence including a single eye blink or any number of eye blinks within a predetermined eye blink time window representing a maximum time duration configured to the sensor circuitry for an eye blink sequence to be sensed.

Example 73 includes the subject matter of Example 69, further including determining a pattern of the plurality of patterns of sensor input, wherein the information based on the haptic input corresponds to information based on the pattern.

Example 74 includes the subject matter of Example 73, further including: performing a correlation of the pattern with its corresponding one of the plurality of ultrasound exam functions; deriving from the correlation the information based on the pattern; and
sending the information based on the pattern to the computing system.

Example 75 includes the subject matter of Example 74, further including storing information on a correlation between each pattern of sensor input of the plurality of patterns of sensor input, and corresponding ones of the plurality of ultrasound exam functions.

Example 76 includes the subject matter of Example 59, further including sending signals for wireless transmission by a wireless transceiver.

Example 77 includes an apparatus comprising means for performing the method of any one of Examples 22-39 and 59-76.

Example 78 includes one or more computer-readable media comprising a plurality of instructions stored thereon that, when executed, cause one or more processors to perform the method of any one of Examples 22-39 and 59-76.

Example 79 includes an imaging device comprising the apparatus of any one of Examples 1-21 and 40-58, and further including the user interface device.

Example 80 includes a product comprising one or more tangible computer-readable non-transitory storage media comprising computer-executable instructions operable to, when executed by at least one computer processor, enable the at least one processor to perform the method of any one of Examples 22-39 and 59-76.

What is claimed is:

1. An ultrasound imaging device including a sensor circuitry and a housing, the sensor circuitry disposed in the housing and coupled thereto to:
sense a haptic input at a surface area of the housing; and
send to a computing system information based on the haptic input to cause an ultrasound exam function to be executed at the computing system, the ultrasound exam function to control an ultrasound image on a display of the computing system.

2. The ultrasound imaging device of claim 1, wherein the sensor circuitry is further to send information based on the haptic input to a sensor signal processing circuitry, the sensor signal processing circuitry to determine a correlation between the sensed haptic input and one or more ultrasound exam functions to be executed at the computing system.

3. The ultrasound imaging device of claim 1, wherein the sensor circuitry includes an accelerometer.

4. The ultrasound imaging device of claim 3, wherein the sensor circuitry further includes a gyroscope.

5. The ultrasound imaging device of claim 4, wherein the sensor circuitry includes a sensor device and a sensor processing circuitry coupled to the sensor device, the sensor device including the accelerometer and the gyroscope, and the sensor processing circuitry to fuse signals corresponding to raw accelerometer data from the accelerometer with signals corresponding to raw gyroscope data from the gyroscope by processing the raw accelerometer data and the raw gyroscope data to generate signals therefrom corresponding to an inertial status of the ultrasound imaging device.

6. The ultrasound imaging device of claim 5, wherein the inertial status of the ultrasound imaging device includes information based on at least one of whether the ultrasound imaging device is stationary, an angular tilt of the ultrasound imaging device with respect to a skin surface of a body being examined, an angular velocity of the ultrasound imaging device with respect to the skin, a position of the ultrasound imaging device on the skin, or a linear velocity of the ultrasound imaging device.

7. The ultrasound imaging device of claim 5, the ultrasound imaging device to receive signals based on the inertial status and to communicate feedback to a user of the ultrasound imaging device derived from the signals based on the inertial status, the feedback corresponding to an adjustment of an ultrasound examination by the user.

8. The ultrasound imaging device of claim 1, wherein the haptic input includes one or more taps on a surface of the housing.

9. The ultrasound imaging device of claim 1, wherein the haptic input includes aerial motion of the ultrasound imaging device.

10. The ultrasound imaging device of claim 1, the sensor circuitry to sense sensor input corresponding to a plurality of patterns of sensor input, each pattern of sensor input including, in a predetermined order, one or more of: the haptic input, an eye tracking input or a voice command input, each pattern of sensor input associated with a corresponding one of a plurality of ultrasound exam functions.

11. The ultrasound imaging device of claim 10, wherein the ultrasound exam functions include at least one of freezing and unfreezing the ultrasound image, saving the ultrasound image, taking a snapshot of the ultrasound image, starting and stopping a recording of ultrasound video, adjusting a depth of the ultrasound image or adjusting a gain of the ultrasound image.

12. The ultrasound imaging device of claim 10, the sensor circuitry to further determine a pattern of the plurality of patterns of sensor input, wherein the information based on the haptic input corresponds to information based on the pattern.

13. A method to be performed at an ultrasound imaging device, the method including:
sensing a haptic input at a surface area of a housing of the ultrasound imaging device; and
sending to a computing system information based on the haptic input to cause an ultrasound exam function to be executed at the computing system, the ultrasound exam function to control an ultrasound image on a display of the computing system.

14. The method of claim 13, further including sensing sensor input corresponding to a plurality of patterns of sensor input, each pattern of sensor input including, in a predetermined order, one or more of: the haptic input, an eye tracking input or a voice command input, each pattern of sensor input associated with a corresponding one of a plurality of ultrasound exam functions.

15. The method of claim 14, wherein the haptic input includes any one of a plurality of permutations of one or more tap sequences, a tap sequence including a single tap or any number of taps within a predetermined tap sequence time window representing a maximum time duration configured for a tap sequence to be sensed.

16. The method of claim 14, wherein the eye tracking input includes an eye blink sequence including a single eye blink or any number of eye blinks within a predetermined eye blink time window representing a maximum time duration configured for an eye blink sequence to be sensed.

17. The method of claim 14, further including determining a pattern of the plurality of patterns of sensor input, wherein the information based on the haptic input corresponds to information based on the pattern.

18. The method of claim 17, further including:
performing a correlation of the pattern with its corresponding one of the plurality of ultrasound exam functions;
deriving from the correlation the information based on the pattern; and
sending the information based on the pattern to the computing system.

19. The method of claim 18, further including storing information on a correlation between each pattern of sensor input of the plurality of patterns of sensor input, and corresponding ones of the plurality of ultrasound exam functions.

20. The method of claim 13, further including wirelessly communicating the information to the computing system.

21. One or more tangible non-transitory computer-readable storage media comprising a plurality of instructions stored thereon that, when executed, cause one or more processors of an ultrasound imaging device to perform operations including:
sensing a haptic input at a surface area of a housing of the ultrasound imaging device; and
sending to a computing system information based on the haptic input to cause an ultrasound exam function to be executed at the computing system, the ultrasound exam function to control an ultrasound image on a display of the computing system.

22. The storage media of claim 21, further including sending information based on the haptic input to a sensor signal processing circuitry, the sensor signal processing circuitry to determine a correlation between the sensed haptic input and one or more ultrasound exam functions to be executed at the computing system.

23. The storage media of claim 21, further including fusing signals corresponding to raw accelerometer data from the accelerometer with signals correspond to raw gyroscope data from the gyroscope by processing the raw accelerometer data and the raw gyroscope data to generate signals therefrom corresponding to an inertial status of the ultrasound imaging device.

24. The storage media of claim 23, wherein the inertial status of the ultrasound imaging device includes information based on at least one of whether the ultrasound imaging device is stationary, an angular tilt of the ultrasound imaging device with respect to a skin surface of a body being examined, an angular velocity of the ultrasound imaging device with respect to the skin, a position of the ultrasound imaging device on the skin, or a linear velocity of the ultrasound imaging device.

25. The storage media of claim 24, further including receiving signals based on the inertial status and communicating feedback to a user of the ultrasound imaging device derived from the signals based on the inertial status, the feedback corresponding to an adjustment of an ultrasound examination by the user.
